(19) United States
(12) Patent Application Publication
(10) Pub. No.: US 2026/0358316 A1
(43) Pub. Date: Nov. 12, 2026

(54) DOMESTIC APPLIANCE AND AN AIR QUALITY INDEX

(71) Applicant: Haier US Appliance Solutions, Inc., Wilmington, DE (US)

(72) Inventors: Paul Bryan Cadima, Crestwood, KY (US); Caleb Farrel, Jeffersonville, IN (US)

(21) Appl. No.: 19/199,464

(22) Filed: May 6, 2025

(51) Int. Cl. G01N 33/00; F24C 15/20; G01K 3/00

(52) U.S. Cl. CPC G01N 33/0063; F24C 15/2021; G01K 3/005

(57) ABSTRACT

A domestic appliance may include a cabinet provided within a room. The domestic appliance may include a controller mounted to the cabinet. The controller operably coupled with an air quality sensor positioned within the room. The controller configured for: accessing air quality data corresponding to a target gas within the room from the air quality sensor, determining a reference sensor output variable based on the air quality data and a predetermined time period, determining an air quality index value based on a contemporary sensor output variable and a reference sensor output variable, detecting an air quality event within the room, and pausing the determination of the reference sensor output variable in response to detecting the air quality event within the room.

### FIELD OF THE DISCLOSURE

The present subject matter relates generally to a domestic appliance, and more particularly to systems and methods of operating a domestic appliance.

### BACKGROUND OF THE DISCLOSURE

Many households contain systems or assemblies for improving the quality of ambient air within a room. For instance, kitchen appliances such as ovens, microwaves, cooktops, or the like include ventilation systems, such as range hoods to selectively circulate air through the kitchen or externally exhaust air during or after cooking operations.

Often, existing systems or assemblies utilize sensors for monitoring the quality of the air within the room. Specifically, such sensors can be configured for monitoring relative concentrations of volatile organic compounds (VOCs) within the room. However, drawbacks related to control methods for such sensors exist that can lead to inaccurate or unreliable monitoring of such relative concentrations of VOCs within the room.

Accordingly, systems and methods that obviate one or more of the above-mentioned drawbacks would be beneficial.

### BRIEF DESCRIPTION OF THE DISCLOSURE

Aspects and advantages of the invention will be set forth in part in the following description, or may be obvious from the description, or may be learned through practice of the invention.

In one exemplary aspect of the present disclosure, a domestic appliance is provided. The domestic appliance may include a cabinet provided within a room. The domestic appliance may include a controller mounted to the cabinet. The controller operably coupled with an air quality sensor positioned within the room. The controller configured for: accessing air quality data corresponding to a target gas within the room from the air quality sensor, determining a reference sensor output variable based on the air quality data and a predetermined time period, determining an air quality index value based on a contemporary sensor output variable and a reference sensor output variable, detecting an air quality event within the room, and pausing the determination of the reference sensor output variable in response to detecting the air quality event within the room.

In another exemplary aspect of the present disclosure, a method for operating a domestic appliance is provided. The domestic appliance may include a cabinet provided within a room and a controller mounted to the cabinet. The controller operably coupled with an air quality sensor positioned within the room. The method may include accessing air quality data corresponding to a target gas within the room from the air quality sensor. The method may include determining a reference sensor output variable based on the air quality data and a predetermined time period. The method may include determining an air quality index value based on a contemporary sensor output variable and a reference sensor output variable. The method may include detecting an air quality event within the room. The method may include pausing the determination of the reference sensor output variable in response to detecting the air quality event within the room.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a front view of a kitchen hub according to one or more exemplary embodiments of the present subject matter.

FIG. 2 is a partial perspective view of an over-the-range microwave with ventilation according to one or more exemplary embodiments of the present subject matter.

FIG. 3 provides a flow chart illustrating a method of operating an appliance according to exemplary embodiments of the present disclosure.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components or systems. For example, the approximating language may refer to being within a 10 percent margin (i.e., including values within ten percent greater or less than the stated value). In this regard, for example, when used in the context of an angle or direction, such terms include within ten degrees greater or less than the stated angle or direction (e.g., “generally vertical” includes forming an angle of up to ten degrees in any direction, such as, clockwise or counterclockwise, with the vertical direction V).

The word “exemplary” is used herein to mean “serving as an example, instance, or illustration.” In addition, reference to “an embodiment” or “one embodiment” does not necessarily refer to the same embodiment, although it may. Any implementation described herein as “exemplary” or “an embodiment” is not necessarily to be construed as preferred or advantageous over other implementations.

Except as explicitly indicated otherwise, recitation of a singular processing element (e.g., “a controller,” “a processor,” “a microprocessor,” etc.) is understood to include more than one processing element. In other words, “a processing element” is generally understood as “one or more processing element.” Furthermore, barring a specific statement to the contrary, any steps or functions recited as being performed by “the processing element” or “said processing element” are generally understood to be capable of being performed by “any one of the one or more processing elements.” Thus, a first step or function performed by “the processing element” may be performed by “any one of the one or more processing elements,” and a second step or function performed by “the processing element” may be performed by “any one of the one or more processing elements and not necessarily by the same one of the one or more processing elements by which the first step or function is performed.” Moreover, it is understood that recitation of “the processing element” or “said processing element” performing a plurality of steps or functions does not require that at least one discrete processing element be capable of performing each one of the plurality of steps or functions.

Existing systems for monitoring indoor air quality often utilize air quality sensors to monitor air quality within a room. For example, the air quality sensors may be any suitable sensor, such as a metal oxide sensor, for monitoring concentrations of VOCs within a room. Such air quality sensors are configured to only determine a change in VOC concentrations compared to a recent history of VOC concentrations. However, many challenges exist with such existing systems for monitoring indoor air quality.

One drawback to relying on historical VOC concentrations is that if ambient conditions within the room are such that VOC concentrations remain elevated for relatively long periods, the air quality sensors will eventually shift to recognize these elevated levels as new reference levels. Thus, under certain conditions, the air quality sensors may report normal relative VOC concentrations when the levels are in actuality relatively high. For example, in instances where there is prolonged cooking activity, such as when a user cans foods for extended periods throughout an entire day or during holidays, catering events, or the like when a user cooks for extended periods throughout an entire day, the monitored VOC concentrations will climb with time until the new reference value is much higher than typical. Thus, at later times, a controller that relies on the air quality sensor to determine a response action to increase VOC concentrations within the room may determine no action is required despite their being extended periods of elevated VOC concentrations.

More precise systems and methods, such as gas chromatography, which can measure VOC concentrations in absolute terms and resolve different compound concentrations are known. However, such systems and methods are expensive, require reference gases, and are often only suitable for laboratory settings.

Notably, aspects of the present subject matter provide systems and methods of operating a domestic appliance to establish an air quality index value corresponding to relative concentrations of VOCs that may be present within a room. In particular, aspects of the present subject matter utilize the detection of events (e.g., cooking events, a rate of change of the sensor exceeding a threshold, or the like) to provide improved monitoring of air quality within a room. In particular, when conditions are expected to reflect normal environments, systems and methods of the present subject matter may continuously monitor and log an air quality index value. When events are detected, use of the monitored and logged air quality index may be paused or suspended. In this regard, the influence of events, such as cooking events, on the monitoring of the indoor air quality characteristics may advantageously be reduced or mitigated (e.g., when compared to existing systems, such as described above).

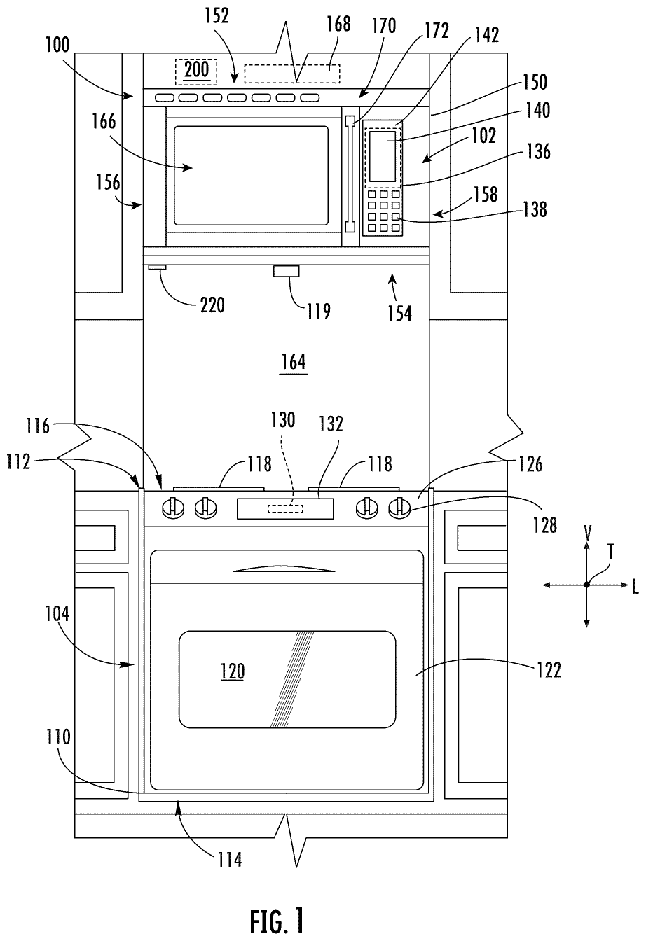

Referring now to the figures, FIG. 1 provides a front view of a kitchen hub 100 according to exemplary embodiments of the present disclosure. Kitchen hub 100 may generally include an over-the-range (OTR) microwave appliance 102 that can be positioned or mounted above a cooktop appliance or range 104. However, it should be appreciated that the present subject matter is not limited to the specific appliances disclosed, and the specific appliance configurations are not intended to limit the scope of the present subject matter in any manner.

As shown in FIG. 1, kitchen hub 100 defines a vertical direction V, a lateral direction L, and a transverse direction T. The vertical, lateral, and transverse directions are mutually perpendicular and form an orthogonal direction system. As used herein, this coordinate system applies equally to both microwave appliance 102 and range 104 and will thus be used interchangeably to describe both appliances and their positions relative to each other.

Range 104 may include a chassis or cabinet 110 that extends along the vertical direction V between a top portion 112 and a bottom portion 114, along the lateral direction L between a left side portion and a right side portion, and along the traverse direction T between a front portion and a rear portion. Range 104 may include a cooktop surface 116 having one or more heating elements 118 for use in, for example, heating or cooking operations. In exemplary embodiments, cooktop surface 116 is constructed with ceramic glass. In other embodiments, however, cooktop surface 116 may include another suitable material, such as a metallic material (e.g., steel) or another suitable non-metallic material. Heating elements 118 may be various sizes and may employ any suitable method for heating or cooking an object, such as a cooking utensil (not shown), and its contents.

In some embodiments, the range 104, and more particularly, a controller associated with the range 104 (e.g., controller 130 described in more detail below) may monitor an operational state of the range 104. The operational state may include or correspond to an activation state of the range 104, such as an activation state of the heating element 118. For example, the operational state may include or correspond to a variable operational state of the heating elements 118, such as a high operational state, a medium operational state, a low operational state, or any other suitable operational state for the heating elements 118 of the range 104. In some embodiments, the operational state of the range 104 may be based on a position of a control knob, such as the input components or the controls 128 described below, associated with the heating elements 118. In this regard, the controller associated with the range 104 may monitor a position of the control knobs to detect when an operational state of the range 104 changes, such as when the heating elements 118 are activated. As will be appreciated in more detail below, the controllers monitoring of the operational state of the range 104 may be utilized in detecting an air quality event, such as a cooking event.

In some embodiments, range 104 includes an insulated cabinet 110 that defines a cooking chamber 120 selectively covered by a door 122. One or more heating elements (not shown, e.g., top broiling elements or bottom baking elements) may be enclosed within cabinet 110 to heat cooking chamber 120. Heating elements within cooking chamber 120 may be provided as any suitable element for cooking the contents of cooking chamber 120, such as an electric resistive heating element, a gas burner, a microwave element, a halogen element, etc. Thus, cooktop appliance 104 may be referred to as an oven range appliance. As will be understood by those skilled in the art, cooktop appliance or range 104 is provided by way of example only, and the present subject matter may be used in the context of any suitable cooking appliance, such as a double oven range appliance or a standalone cooktop (e.g., fitted integrally with a surface of a kitchen counter). Thus, the example embodiments illustrated in figures are not intended to limit the present subject matter to any particular cooking chamber or heating element configuration, except as otherwise indicated.

As illustrated, a user interface (or user interface panel) 126 may be provided on range 104. Although shown at front portion of range 104, another suitable location or structure (e.g., a backsplash) for supporting user interface panel 126 may be provided in alternative embodiments. In some embodiments, user interface panel 126 includes input components or controls 128, such as one or more of a variety of electrical, mechanical, or electro-mechanical input devices. Controls 128 may include, for example, rotary dials, knobs, push buttons, and touch pads. A controller 130 is in communication with user interface panel 126 and controls 128 through which a user may select various operational features and modes and monitor progress of range 104. In additional or alternative embodiments, user interface panel 126 includes a display component 132, such as a digital or analog display in communication with a controller 130 and configured to provide operational feedback to a user. In certain embodiments, user interface panel 126 represents a general purpose I/O (“GPIO”) device or functional block.

As shown, controller 130 is communicatively coupled (e.g., in operative communication) with user interface panel 126, controls 128, and display 132. Controller 130 may also be communicatively coupled with various operational components of range 104 as well, such as heating elements (e.g., 118), sensors, etc. Input/output (“I/O”) signals may be routed between controller 130 and the various operational components of range 104. Thus, controller 130 may selectively activate and operate these various components. Various components of range 104 are communicatively coupled with controller 130 via one or more communication lines such as, for example, conductive signal lines, shared communication busses, or wireless communications bands.

In some embodiments, controller 130 includes one or more memory devices and one or more processors. The processors can be any combination of general or special purpose processors, CPUs, or the like that can execute programming instructions or control code associated with operation of range 104. The memory devices (e.g., memory) may represent random access memory such as DRAM or read only memory such as ROM or FLASH. In one embodiment, the processor executes programming instructions stored in memory. The memory may be a separate component from the processor or may be included onboard within the processor. Alternatively, controller 130 may be constructed without using a processor, for example, using a combination of discrete analog or digital logic circuitry (such as switches, amplifiers, integrators, comparators, flip-flops, AND/OR gates, and the like) to perform control functionality instead of relying upon software.

In certain embodiments, controller 130 includes a network interface such that controller 130 can connect to and communicate over one or more networks with one or more network nodes. Controller 130 may also include one or more transmitting, receiving, or transceiving components for transmitting/receiving communications with other devices communicatively coupled with range 104. Additionally, or alternatively, one or more transmitting, receiving, or transceiving components can be located off board controller 130. Generally, controller 130 may be positioned in any suitable location throughout range 104. For example, controller 130 may be located proximate to user interface panel 126 toward front portion of range 104. In optional embodiments, controller 130 is in operable communication with a controller 142 (described below) of microwave appliance 102 (e.g., through one or more wired or wireless channels).

Referring still to FIG. 1, OTR microwave appliance 102 may include a control panel 136 that may represent a general-purpose Input/Output (“GPIO”) device or functional block for microwave appliance 102. In some embodiments, control panel 136 may include or be in operative communication with one or more user input devices 138, such as one or more of a variety of digital, analog, electrical, mechanical, or electro-mechanical input devices including rotary dials, control knobs, push buttons, toggle switches, selector switches, and touch pads. Additionally, microwave appliance 102 may include a display 140, such as a digital or analog display device generally configured to provide visual feedback regarding the operation of microwave appliance 102. For example, display 140 may be provided on control panel 136 and may include one or more status lights, screens, or visible indicators. According to exemplary embodiments, user input devices 138 and display 140 may be integrated into a single device, e.g., including one or more of a touchscreen interface, a capacitive touch panel, a liquid crystal display (LCD), a plasma display panel (PDP), a cathode ray tube (CRT) display, or other informational or interactive displays.

Microwave appliance 102 may further include or be in operative communication with a processing device or a controller 142 that may be generally configured to facilitate appliance operation. In this regard, control panel 136, user input devices 138, and display 140 may be in communication with controller 142 such that controller 142 may receive control inputs from user input devices 138, may display information using display 140, and may otherwise regulate operation of microwave appliance 102. For example, signals generated by controller 142 may operate microwave appliance 102, including any or all system components, subsystems, or interconnected devices, in response to the position of user input devices 138 and other control commands. Control panel 136 and other components of microwave appliance 102 may be in communication with controller 142 via, for example, one or more signal lines or shared communication busses. In this manner, Input/Output (“I/O”) signals may be routed between controller 142 and various operational components of microwave appliance 102.

As used herein, the terms “processing device,” “computing device,” “controller,” or the like may generally refer to any suitable processing device, such as a general or special purpose microprocessor, a microcontroller, an integrated circuit, an application specific integrated circuit (ASIC), a digital signal processor (DSP), a field-programmable gate array (FPGA), a logic device, one or more central processing units (CPUs), a graphics processing units (GPUs), processing units performing other specialized calculations, semiconductor devices, etc. In addition, these “controllers” are not necessarily restricted to a single element but may include any suitable number, type, and configuration of processing devices integrated in any suitable manner to facilitate appliance operation. Additionally or alternatively, controller 142 may be constructed without using a microprocessor, e.g., using a combination of discrete analog or digital logic circuitry (such as switches, amplifiers, integrators, comparators, flip-flops, AND/OR gates, and the like) to perform control functionality instead of relying upon software.

Controller 142 may include, or be associated with, one or more memory elements or non-transitory computer-readable storage mediums, such as RAM, ROM, EEPROM, EPROM, flash memory devices, magnetic disks, or other suitable memory devices (including combinations thereof). These memory devices may be a separate component from the processor or may be included onboard within the processor. In addition, these memory devices can store information or data accessible by the one or more processors, including instructions that can be executed by the one or more processors. It should be appreciated that the instructions may be software written in any suitable programming language or can be implemented in hardware. Additionally, or alternatively, the instructions may be executed logically or virtually using separate threads on one or more processors.

For example, controller 142 may be operable to execute programming instructions or micro-control code associated with an operating cycle of appliance 102. In this regard, the instructions may be software or any set of instructions that when executed by the processing device, cause the processing device to perform operations, such as running one or more software applications, displaying a user interface, receiving user input, processing user input, etc. Moreover, it should be noted that controller 142 as disclosed herein is capable of and may be operable to perform any methods, method steps, or portions of methods as disclosed herein. For example, in some embodiments, methods disclosed herein may be embodied in programming instructions stored in the memory and executed by controller 142.

The memory devices may also store data that can be retrieved, manipulated, created, or stored by the one or more processors or portions of controller 142. The data can include, for instance, data to facilitate performance of methods described herein. The data can be stored locally (e.g., on controller 142) in one or more databases or may be split up so that the data is stored in multiple locations. In addition, or alternatively, the one or more database(s) can be connected to controller 142 through any suitable network(s), such as through a high bandwidth local area network (LAN) or wide area network (WAN). In this regard, for example, controller 142 may further include a communication module or interface that may be used to communicate with one or more other component(s) of microwave appliance 102, controller 142, an external appliance controller, or any other suitable device, e.g., via any suitable communication lines or network(s) and using any suitable communication protocol. The communication interface can include any suitable components for interfacing with one or more network(s), including for example, transmitters, receivers, ports, controllers, antennas, or other suitable components.

As noted above, microwave appliance 102 may be positioned or mounted above range 104 (e.g., as an OTR microwave). Specifically, a cabinet 150 of OTR microwave appliance 102 may be positioned above range 104 along the vertical direction V.

In some embodiments, the cabinet 150 is an insulated cabinet. As shown, cabinet 150 of microwave appliance 102 includes a plurality of outer walls and when assembled, microwave appliance 102 generally extends along the vertical direction V between a top end 152 and a bottom end 154, along the lateral direction L between a first side end 156 and a second side end 158, and along the transverse direction T between a front end 157 and a rear end 159. The cabinet 150 may include a first exterior side panel 176 at the first side 156 laterally spaced from second external side panel 178 at the second side 158. In addition, the cabinet 150 may include a front panel 180 at the front end 157 and a back panel 182 at the back end 159.

In some embodiments, cabinet 150 is spaced apart from cooktop surface 116 along the vertical direction V. An open region 164 may thus be defined along the vertical direction V between cooktop surface 116 and bottom end 154 of cabinet 150. Although a generally rectangular shape is illustrated, any suitable shape or style may be adapted to form the structure of cabinet 150. Within cabinet 150, an internal liner of cabinet 150 defines a cooking chamber 166 for receipt of food items for cooking.

A microwave oven enclosure 162 may be supported within cabinet 150. Microwave oven enclosure 162 may include a first side wall 190 laterally spaced from the first external side panel 176. The first side wall 190 defines a first fan inlet 192 formed as an opening in the first side wall 190. As illustrated, the first fan inlet 192 may be formed by removing a portion of the first side wall 190. In other embodiments, the first fan inlet 192 may be a series of smaller openings, for example holes or slots, formed in the first side wall 190 to allow a flow of air to pass.

Microwave oven enclosure 162 includes a second side wall 194 laterally spaced from the second external side panel 178. The second side wall 194 also defines a fan inlet, second fan inlet 196 formed, as above, as an opening in the second side wall 194. Also as above, the second fan inlet 196 may be formed by removing a portion of the second side wall 194 or, in some embodiments, may be a series of smaller openings, for example holes or slots, formed in the second side wall 194 to allow a flow of air to pass. The microwave oven enclosure 162 may also include a bottom panel 198 joining the first side wall 190 and the second side wall 194 at a lower end of each wall.

Microwave appliance 102 is generally configured to heat articles (e.g., food or beverages) within cooking chamber 166 using electromagnetic radiation. Microwave appliance 102 may include various components which operate to produce electromagnetic radiation, as is generally understood. For example, microwave appliance 102 may include a heating assembly 168 in mechanical space 200, the heating assembly 168 having a magnetron (e.g., a cavity magnetron), a high voltage transformer, a high voltage capacitor, and a high voltage diode, as is understood. The transformer may provide energy from a suitable energy source (such as an electrical outlet) to the magnetron. The magnetron may convert the energy to electromagnetic radiation, specifically microwave radiation. The capacitor generally connects the magnetron and transformer, such as via high voltage diode, to a chassis. Microwave radiation produced by the magnetron may be transmitted through a waveguide to cooking chamber 166.

The structure and intended function of microwave ovens or appliances are generally understood by those of ordinary skill in the art and are not described in further detail herein.

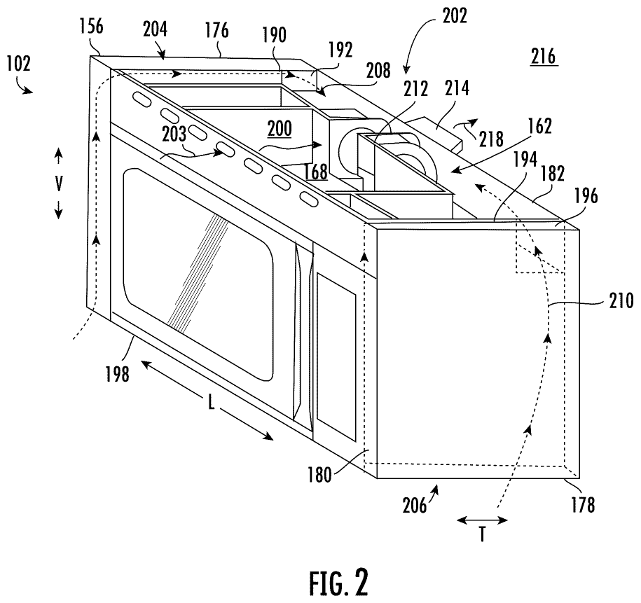

Microwave appliance 102 may include a door assembly 170 that is movably mounted (e.g., rotatably attached) to cabinet 150 in order to permit selective access to cooking chamber 166. Specifically, door assembly 170 may move between an open position and a closed position (e.g., FIGS. 1 and 2). The open position permits access to cooking chamber 166 while the closed position restricts access to cooking chamber 166. A handle 172 may be mounted to or formed on door assembly 170 to assist a user with opening and closing door assembly 170.

Kitchen hub 100 may include a ventilation system 202. Ventilation system 202 may include an air handler 212 for circulating or exhausting air, for instance, within or out of the room the kitchen hub 100 is located in. As shown, the air handler 212 may be provided within microwave appliance 102. As such, microwave appliance 102 may be referred to as a casing of air handler 212. 168

As should be appreciated, the reference to the microwave appliance 102 in relation to the ventilation system 202 is for illustration, not limitation. Any appliance, for example a kitchen appliance, may be paired with the ventilation system 202 to yield an over the range kitchen appliance in accordance with this disclosure. For example, a radiant heat oven, a combination cooking mode oven, or a communications or entertainment hub may be paired with the ventilation system 202 without departing the scope of this disclosure.

As described above and illustrated at least in FIG. 2, the first side wall 190, the first external side panel 176, the front panel 180, the back panel 182, and the top panel 184 may define a first duct 204. Similarly, the second side wall 194, the second external side panel 178, the front panel 180, the back panel 182, and the top panel 184 may define a second duct 206. A first ducts 204 and a second duct 206 may be open at the bottom 154 of the cabinet 150 and closed at the upper end by top panel 184. First fan inlet 192 may provide an outlet to first duct 204 and together, first duct 204 and first fan inlet 192 may define a first exhaust path 208. Similarly, second fan inlet 196 may provide an outlet to second duct 206, and second duct 206 and second fan inlet 196 may cooperate to define a second exhaust path 210.

First exhaust path 208 and second exhaust path 210 may be in fluid communication with air handler 212 mounted in cabinet 150. As illustrated, air handler 212 may be positioned adjacent to the back panel 182 of cabinet 150. The location of air handler 212 may be any suitable location facilitating a flow of air through the first exhaust path 208 or the second exhaust paths 210. In an embodiment, air handler 212 draws air in from the first duct 204 and the second duct 206 forming first exhaust path 208 and second exhaust path 210, respectively. The first exhaust path 208 and the second exhaust path 210 may be combined at air handler 212 to form exhaust 218 which, in an embodiment, flows through an exhaust vent 214 to the room 216 in which the kitchen hub 100 is located within. Accordingly, the combined exhaust 218 may include first duct 204 and second duct 206, first fan inlet 192 and second fan inlet 196, fan 212, and the exhaust vent 214.

According to the illustrated exemplary embodiment, air handler 212 is an axial fan positioned within casing. However, it should be appreciated that according to alternative embodiments, air handler 212 may be positioned at any other suitable location and may be any other suitable fan type, such as a tangential fan, a centrifugal fan, etc.

In addition, according to an exemplary embodiment, air handler 212 is a variable speed fan such that it may rotate at different rotational speeds, thereby generating different air flow rates. In this manner, the amount of air circulated through exhaust 218 may be continuously and precisely regulated. Moreover, by pulsing the operation of air handler 212 or throttling air handler 212 between different rotational speeds, the flow of air through exhaust 218 may have a different flow velocity or may generate a different flow pattern within cabinet 150.

In some embodiments, the kitchen hub 100 includes one or more temperature sensors 119 for detecting thermal events within the room 216. For example, as shown in FIG. 1, the kitchen hub 100 includes a temperature sensor 119 positioned at or below the microwave appliance 102. As used herein, “temperature sensor” or the equivalent is intended to refer to any suitable type of temperature measuring system or device for measuring or detecting thermal events within the room. For example, the temperature sensors 119 may be any suitable type of temperature sensor, such as a thermistor, a thermocouple, a resistance temperature detector, a semiconductor-based integrated circuit temperature sensor, etc., for measuring the temperature of ambient air within the room 216. As another example, the temperature sensors 119 may include or be provided as an infrared sensor configured for detecting thermal events at a predetermined location, such as at the range 104. In addition, temperature sensors 119 may output a signal, such as a voltage, to a controller, such as the controller 130 or the controller 142, that is proportional to, or indicative of the temperature being measured.

Although exemplary positioning of temperature sensors is described herein, it should be appreciated that kitchen hub 100 may include any other suitable number, type, and position of temperature or other sensors according to alternative embodiments.

Kitchen hub 100 may further include at least one air quality sensor 220 (e.g., in electrical or wireless communication with a controller (e.g., the controller 130, the controller 142, or any other suitable controller). During use, the air quality sensor 220 may communicate with the controller to send information to the controller. Generally, the at least one air quality sensor 220 is configured to monitor one or more air-quality characteristic of air within a room 216 that the kitchen hub 100 is located within. Air quality sensor 220 may be located at any appropriate location within the room 216. For example, as shown in FIG. 1, the air quality sensor 220 may be provided below microwave appliance 102. As another example, the air quality sensor 220 may be positioned within the ventilation system 202.

As mentioned above, the air quality sensor 220 may be configured to monitor air quality data corresponding to one or more air-quality characteristic of a target gas (e.g., toxic gases, harmful emissions, pollutants, or any other suitable volatile organic compound (VOC)) within the room 216. The air-quality characteristics of the target gas may include or be provided as relative levels of the target gas within the room. In particular, the air quality sensor 220 may detect changes in contemporary (e.g., current) air-quality characteristics in the room 216 relative to historical air-quality characteristics in the room 216. In other words, the air quality sensor 220 may detect air-quality characteristics in relative terms rather than absolute terms. As will be appreciated in more detail below, the relative change may be described as an air quality index value that may correspond to a relative air quality within the room 216. In some embodiments, the air quality sensor 220 includes or is provided as a volatile organic compound (VOC) sensor (e.g., a metal oxide sensor) for monitoring a relative VOC status (e.g., a relative VOC concentration or level) within the room 216.

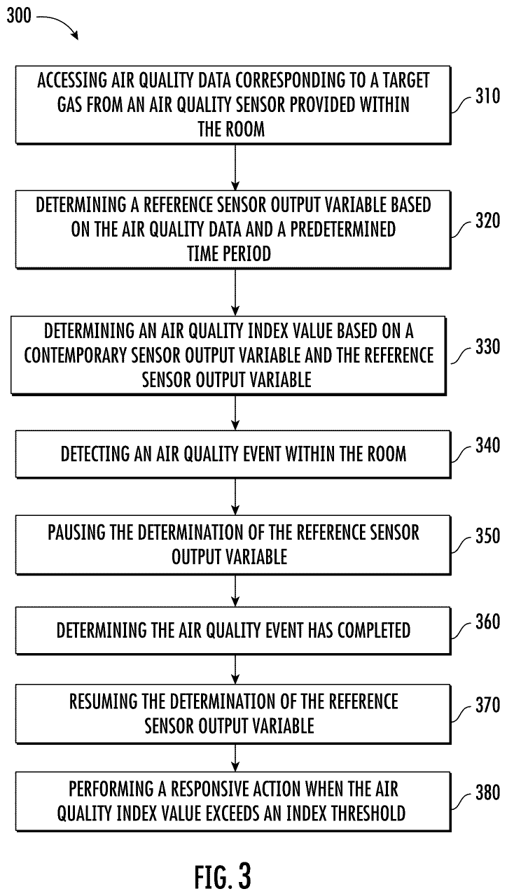

Referring now to FIG. 3, a flow diagram of a method 300 of operating a domestic appliance provided within a room is provided. The method 300 may be used to operate any suitable domestic appliance that may be provided within a room and that may be operably coupled to an air quality sensor. For example, the method 300 may be used to operate the OTR microwave appliance 102 of the kitchen hub 100 that may be operably coupled to the air quality sensor 220, the range 104 of the kitchen hub 100 that may be operably coupled to the air quality sensor 220, or any other suitable domestic appliance that may be provided within the room 216 and operably coupled to the air quality sensor 220. A controller of the domestic appliance may be programmed to implement method 300. For example, the controller 130 or the controller 142 may be programmed to implement method 300.

FIG. 3 depicts steps performed in a particular order for purpose of illustration and discussion. Those of ordinary skill in the art, using the disclosures provided herein, will understand that the steps of any of the methods disclosed herein can be modified, adapted, rearranged, omitted, or expanded in various ways without deviating from the scope of the present disclosure (except as otherwise described).

At 310, the method 300 includes accessing (e.g., receiving, retrieving, or otherwise obtaining) air quality data corresponding to a target gas from an air quality sensor provided within the room. The air quality sensor may be any suitable sensor provided within an area or space in which the domestic appliance is also provided within. The air quality sensor may be configured to detect or identify relative changes in a level of a target gas (e.g., VOCs or toxic gases, such as carbon monoxide, formaldehyde, nitrogen dioxide, ammonia, or the like) within the room. In this regard, the air quality data accessed may include data or information corresponding to relative changes in a level of a target gas within the room. Relative changes in the level of the target gas within the room may include a relative increase or decrease in a contemporary level (e.g., a current concentration or intensity) of the target gas within the room compared to a previous level (e.g., a previous concentration or intensity) of the target gas within the room.

In some embodiments, accessing the air quality data includes receiving an output signal from the air quality sensor. In such embodiments, the air quality data may correspond to a magnitude (e.g., a strength or intensity) of a signal component (e.g., a voltage, current, resistance, or the like) of the output signal. In particular, as the air quality sensor detects relative changes in the level of the target gas within the room, a contemporary magnitude of the signal component of the output signal may change. As an example, when the air quality sensor includes a VOC sensor, such as a MOX sensor, the output signal may include an electrical resistance signal component that corresponds to the electrical resistance of the VOC sensor. In such instances, as the VOC sensor reacts to relative changes in the level of a target gas within the room, a magnitude of the electrical resistance signal component may change (e.g., increase or decrease).

At 320, the method includes determining a reference sensor output variable based on the air quality data (e.g., obtained at 310) and a predetermined time period (e.g., six hours, twelve hours, twenty-four hours, thirty-six hours, or any other suitable time interval). In some embodiments, the controller may utilize a moving average (e.g., a simple moving average or an exponential moving average) to determine the reference sensor output variable. For example, the moving average may be utilized to determine an average magnitude of the signal component across a predetermined time period, such as twelve hours. The moving average may be “moving” in that it is determining or updating (e.g., continuously or at a predetermined interval) the reference sensor output variable as new data is detected (e.g., by the air quality sensor) and accessed (e.g. by the controller of the domestic appliance). In this regard, the reference sensor output variable may include a reference value that corresponds to the average magnitude of the signal component of the output signal across the predetermined time period. As will be appreciated in more detail below, the updating of the reference value of the reference sensor output variable may be paused when an air quality event is detected.

At 330, the method includes determining an air quality index value based on a contemporary sensor output variable and the reference sensor output variable. The air quality index value may be a numerical value that represents a relative change in the level of the target gas within the room. In other words, the air quality index value may be utilized to represent how a current or contemporary level of the target gas in the room is different (e.g., higher or lower), than a previous level of the target gas in the room. In some embodiments, the air quality index value is represented as a numerical value within a predetermined index range. The predetermined index range may be any suitable index range that may represent a relative intensity of a target gas within the room. For example, the predetermined index range may be 1 to 500, 1 to 1000, or the like. In such instances, the predetermined index range may include a base value, for instance, 100, that corresponds to a reference or a normal level of the target gas within the room. In some embodiments, the base value is indicative of the reference sensor output variable. In this regard, an air quality index value that is higher than the base value may represent a relative increase in the level of the target gas within the room, for instance, when compared to the average level of the target gas within the room across the most recent predetermined time period.

In some embodiments, determining the air quality index value includes comparing the contemporary sensor output variable (e.g., the current or most recent level of target gas within the room) and the reference sensor output variable (e.g., the average level of the target gas within the room across the most recent predetermined time period). In particular, the controller may compare the contemporary sensor output variable to the reference sensor output variable to determine the relative change between the variables. The air quality index value may be based on this relative change between the variables. As should be appreciated, the air quality index value may be updated continuously or at a predetermined interval. For instance, the air quality index value may be updated in response to new air quality data being accessed by the controller.

At 340, the method 300 includes detecting an air quality event within the room. The air quality event may be any suitable detectable event, for instance, of a domestic appliance, that may produce a target gas within the room. For example, the air quality event may be a cooking event that may produce a target gas within the room. In some embodiments, detecting the air quality event within the room includes determining a rate of change of the contemporary sensor output variable exceeds a predetermined sensor threshold. For example, when the contemporary sensor output variable increases at a rate that exceeds the predetermined sensor threshold (e.g., a predetermined threshold rate of change for the contemporary sensor output variable), it may be determined that an air quality event is occurring within the room.

Additionally or alternatively, in some embodiments, detecting the air quality event within the room includes detecting a change in operational state of the domestic appliance (e.g., a range). For example, detecting the change in operational state of the domestic appliance may include monitoring a position of input components or controls (e.g., rotary dials, knobs, push buttons, or touch pads) of the domestic appliance. The position of the input components or controls may correspond to, or indicate, the operational state of the domestic appliance. For example, when the domestic appliance is a range, the controller may monitor the position of the control knobs associated with the heating elements. In this regard, when a position of the control knob is changed, such as to turn on a gas heating element, the controller may detect an air quality event within the room.

Additionally or alternatively, in some embodiments, detecting the air quality event within the room includes determining, with a temperature sensor positioned with the room, a temperature measurement exceeds a predetermined temperature threshold. As mentioned above, the controller may be configured to access (e.g., retrieve, receive, or otherwise obtain) temperature data from a temperature sensor provided within the room. For example, the temperature sensor may be any suitable temperature sensor configured to sense or detect a thermal event within the room. For instance, the temperature sensor may be configured to detect changes in the ambient temperature within the room, changes in temperature at predetermined locations within the room (e.g., at a range), or the like. The predetermined temperature threshold may correspond to any suitable temperature threshold that may indicate an air quality event within the room. For example, the predetermined temperature threshold may correspond to any suitable absolute temperature measurement that when exceeded indicates an air quality event within the room. As another example, the predetermined temperature threshold may correspond to a rate of change in temperature measurement across any suitable time interval that may indicate an air quality event, such as a cooking event, is taking place. In such instances, the predetermined temperature threshold may correspond to the temperature measurements of ambient air within the room increasing by five degrees Fahrenheit (5 °F) over a one minute time interval. As another example, the predetermined temperature threshold may correspond to the temperature measurements of ambient air within the room increasing by ten degrees Fahrenheit (10 °F) over a one minute time interval.

Additionally or alternatively, in some embodiments, the domestic appliance may be in operative communication with one or more connected appliances (e.g., one or more appliances that may in operative communication with the domestic appliance, for instance, via a wireless network). In such embodiments, detecting the air quality event includes receiving an air quality signal from the one or more connected appliances. In some embodiments, the air quality signal is indicative of a temperature measurement of the one or more connected appliances exceeding a temperature threshold. In some other embodiments, the air quality signal is indicative of an initiation of an operating cycle (e.g., a cooking cycle) for the one or more connected appliances.

At 350, the method 300 includes pausing the determination of the reference sensor output variable in response to detecting the air quality event. In some embodiments, pausing the determination of the reference sensor output variable includes pausing the determination or the updating of the reference sensor output variable until it is determined (e.g., at the controller) that the air quality event has completed. As mentioned above, the reference sensor output variable may be determined continuously or at a predetermined interval via a moving average. In this regard, pausing the determination of the reference sensor output variable may include pausing the moving of the window for the moving average until completion of the air quality event. Notably, pausing the determination of the reference sensor output variable advantageously mitigates or minimizes the influence that air quality events may have on the determination of the air quality index value (e.g., when compared to existing systems and methods). For example, during an air quality event, the contemporary sensor output variable may be compared to the previous, or most recent, reference sensor output variable. In this regard, the air quality index value may still be determined and utilized when determining when to perform a responsive action within the room (e.g., described in more detail below).

At 360, the method 300 includes determining the air quality event has completed. In some embodiments, determining the air quality event has completed includes determining the contemporary sensor output variable is within a predetermined range of the previous or most recent reference sensor output variable prior to the air quality event. For example, the contemporary sensor output variable may be compared (e.g., continuously or at a predetermined interval) to the reference sensor output variable during the air quality event such that it may be determined when the contemporary sensor output variable is within a predetermined range of the reference sensor output variable.

Additionally or alternatively, in some embodiments, determining the air quality event has completed includes receiving data or information from the temperature sensor at the heating element indicating no heating is present at the heating element. For example, when the temperature sensor associated with the heating elements obtains temperature data indicating heating is no longer present is no longer present, it may be determined that the air quality event has completed.

Additionally or alternatively, in some embodiments, determining the air quality event has completed includes receiving a completion signal from the one or more connected appliances. For example, upon completion of the cooking event, such as after the completion of a cooking cycle, after a heating element has been turned off, such as by a user, or the like, the one or more connected appliances may transmit the completion signal corresponding to the completion of the cooking event (e.g., at the one or more connected appliances) to the controller of the domestic appliance.

At 370, the method 300 includes resuming the determination of the reference sensor output variable in response to determining the air quality event has completed. After the air quality event has completed or when the level of the target gas within the room has returned to a nominal level, the determination of the reference sensor output variable may be resumed. In this regard, the controller may determine the moving average of the air quality data such that the reference sensor output variable may be updated (e.g., continuously or at a predetermined interval).

At 380, the method 300 includes performing a responsive action when the air quality index value exceeds an index threshold. In some embodiments, 380 includes monitoring the air quality index value. For instance, the air quality index value may be monitored before, during, or after the detection of an air quality event. Monitoring the air quality index value may include comparing (e.g., continuously or at a predetermined interval) the air quality index value to the index threshold to determine when the air quality index value exceeds the index threshold. In some embodiments, when it is determined that the air quality index value has exceeded the index threshold, 380 includes transmitting an air quality signal to a user interface associated with the domestic appliance. For example, the controller may transmit the air quality signal to a display associated with the domestic appliance. In such instances, the air quality signal may direct the display perform a responsive action, such as displaying a user notification indicating the performance of the responsive action, emitting a light to notify the user of the performance of the responsive action, or the like. As another example, the controller may transmit the air quality signal to an external device (e.g., a smartphone, smartwatch, tablet, or the like) in operative communication with the domestic appliance. In such instances, the air quality signal may, for example, direct the external device to display a user notification indicating the performance of the responsive action.

Additionally or alternatively, in some other embodiments, when it is determined that the air quality index value exceeds the index threshold, 380 may include directing a ventilation system in operative communication with the controller according to a condition cycle. The condition cycle may include activating an air handler of the ventilation system. As described above, the air handler may be a variable speed air handler or fan capable of operating (e.g., rotating) at a plurality of speeds to produce varying airflows. Thus, the initiation of the condition cycle may include activating the air handler at a first predetermined speed to condition the ambient air within the room.

Advantageously, exemplary methods of the present subject matter permit increased data collection or otherwise improve data handling (e.g., when compared to existing methods that do not utilize air quality event detection. In particular, methods in accordance with the present disclosure advantageously detect air quality events and pause the determination of a reference sensor output variable in response to the detection of the air quality event. In this regard, the controller may utilize the detection of the air quality event to improve the determination of the air quality index value as the influence of air quality events, such as prolonged cooking events, may be reduced or mitigated.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A domestic appliance comprising:
a cabinet provided within a room; and
a controller mounted to the cabinet, the controller operably coupled with an air quality sensor positioned within the room, the controller configured to execute instructions that cause the domestic appliance to perform operations, the operations comprising:
accessing air quality data corresponding to a target gas within the room from the air quality sensor,
determining a reference sensor output variable based on the air quality data and a predetermined time period, wherein determining the reference sensor output variable comprises determining the reference sensor output variable via a moving average,
determining an air quality index value based on a contemporary sensor output variable and the reference sensor output variable,
detecting an air quality event within the room, and
pausing the determination of the reference sensor output variable in response to detecting the air quality event within the room, wherein pausing the determination of the reference sensor output variable comprises pausing the moving average in response to detecting the air quality event within the room.

2. The domestic appliance of claim 1, wherein the controller is further configured for:
determining the air quality event has completed following pausing the determination of the reference sensor output variable; and
resuming the determination of the reference sensor output variable in response to determining the air quality event has completed.

3. (canceled)

4. (canceled)

5. The domestic appliance of claim 1, wherein detecting the air quality event with the room comprises:
determining a rate of change of the contemporary sensor output variable exceeds a predetermined sensor threshold.

6. The domestic appliance of claim 1, further comprising:
a temperature sensor positioned within the room, wherein detecting the air quality event within the room comprises:
determining, with the temperature sensor, a temperature measurement exceeds a predetermined temperature threshold.

7. The domestic appliance of claim 1, detecting the air quality event within the room comprises:
receiving an air quality signal indicative of an operating cycle from a connected appliance in operative communication with the controller via a network.

8. The domestic appliance of claim 1, wherein the target gas within the room corresponds to a volatile organic compound.

9. The domestic appliance of claim 1, wherein the controller is further configured for:
performing a responsive action when the air quality index value exceeds an index threshold.

10. The domestic appliance of claim 9, wherein performing the responsive action comprises:
transmitting an air quality signal to a user interface associated with the domestic appliance, the air quality signal including a user notification indicating the performance of the responsive action.

11. A method for operating a domestic appliance comprising a cabinet provided within a room and a controller mounted to the cabinet, the controller operably coupled with an air quality sensor positioned within the room, the method comprising:
accessing air quality data corresponding to a target gas within the room from the air quality sensor;
determining a reference sensor output variable based on the air quality data and a predetermined time period, wherein determining the reference sensor output variable comprises determining the reference sensor output variable via a moving average;
determining an air quality index value based on a contemporary sensor output variable and the reference sensor output variable;
detecting an air quality event within the room; and
pausing the determination of the reference sensor output variable in response to detecting the air quality event within the room, wherein pausing the determination of the reference sensor output variable comprises pausing the moving average in response to detecting the air quality event within the room.

12. The method of claim 11, further comprising:
determining the air quality event has completed following pausing the determination of the reference sensor output variable; and
resuming the determination of the reference sensor output variable in response to determining the air quality event has completed.

13. (canceled)

14. (canceled)

15. The method of claim 11, wherein detecting the air quality event with the room comprises:
determining a rate of change of the contemporary sensor output variable exceeds a predetermined sensor threshold.

16. The method of claim 11, wherein the domestic appliance comprises a temperature sensor positioned within the room,
wherein detecting the air quality event within the room comprises:
determining, with the temperature sensor, a temperature measurement exceeds a predetermined temperature threshold.

17. The method of claim 11, detecting the air quality event within the room comprises:
receiving an air quality signal indicative of an operating cycle from a connected appliance in operative communication with the controller via a network.

18. The method of claim 11, wherein the target gas within the room corresponds to a volatile organic compound.

19. The method of claim 11, further comprising:
performing a responsive action when the air quality index value exceeds an index threshold.

20. The method of claim 19, wherein performing the responsive action comprises:
transmitting an air quality signal to a user interface associated with the domestic appliance, the air quality signal including a user notification indicating the performance of the responsive action.
